(19) United States
(12) Patent Application Publication
(10) Pub. No.: US 2021/0162113 A1
(43) Pub. Date: Jun. 3, 2021

(54) DISPOSABLE IRRIGATION BASIN FOR EXTREMITY PROCEDURES

(71) Applicant: MDM Wound Ventures, Inc., Schertz, TX (US)

(72) Inventor: Francis Derk, San Antonio, TX (US)

(73) Assignee: MDM Wound Ventures, Inc., Schertz, TX (US)

(21) Appl. No.: 16/818,020

(22) Filed: Mar. 13, 2020

Related U.S. Application Data
(60) Provisional application No. 62/818,436, filed on Mar. 14, 2019.

(51) Int. Cl. A61M 3/02; B65D 1/36; B65D 1/40

(52) U.S. Cl. CPC A61M 3/0287; B65D 2501/24; B65D 1/40; B65D 1/36

(57) ABSTRACT

A disposable basin for extremity procedures, irrigation and lavage in the operating room, treatment clinic or ward allows the extremity to rest comfortably, safely, and securely inside the basin, allowing the surgeon, physician, clinician, and or provider ease of access within a contained protective barrier. Contaminated fluid safely and efficiently drains into an integrated suction trough via the intrinsic design of the basin, resulting in decreased exposure to pathogens, containment of contamination, ease of execution and disposal, and cost savings.

### REFERENCE TO RELATED APPLICATIONS

This application claims priority to, and the benefit of, U.S. Provisional Patent Application Ser. No. 62/818,436, filed Mar. 14, 2019, the entire content of which is incorporated herein by reference.

### FIELD OF THE INVENTION

This invention relates generally to wound cleaning and, in particular, to a disposable irrigation basin for extremity procedures.

### BACKGROUND OF THE INVENTION

Irrigation of extremities utilizes large amounts of sterile saline, water, and cleansing agents. Once in contact with infected and/or bloody extremities, unstable basins can splash or “slosh” contaminated fluid onto and under the sterile field, patient, clinicians, and floors and other surfaces of the treatment venue.

Current extremity irrigation trays are unstable and often times require modification, resulting in poor containment of fluid, contamination, and exposure of blood-borne pathogens. Unstable basins can cost time and money by increasing: (1) cleanup costs (cleaning solutions); (2) time management (room turnover); (3) manpower (housekeeping or EMS); and (4) expensive resources (towels, chucks, absorbents, etc.) necessary to contain spillage and decontaminate the patient, clinician and treatment area.

### SUMMARY OF THE INVENTION

This invention provides an improved irrigation basin that allows the extremity to comfortably rest in a stabilized position, restricts fluid within, and allows contaminated fluid to safely and efficiently drain into an integrated suction portal. This results in decrease exposure to pathogens and cleanup costs, safe execution, and quick and thorough disposal.

The disposable irrigation basin disclosed herein allows the extremity to rest securely inside the basin and accommodates a tourniquet. An internal extremity resting slope accommodates up to the distal one third of a lower extremity, depending on what level the physician desires. The basin has a built-in step-off groove (underneath) which anchors to the edge of an OR table, exam chair, exam table, and or any edged examination platform. Once engaged, the distal portion of the basin declines (by design) and is held in place by the body weight of the extremity. As a result, fluids drain into the basin in a “downhill” manner, collect into a series of troughs, and vacate via an integrated suction portal. The contaminated fluid may then travel via suction into FDA approved suction canister(s), thus decreasing contamination. The basin can also be used on any flat surface, does not require suction, and can collect up to 1 liter of irrigation/fluid without spillage.

The inventive wound basin may be used before, during, or following any type of extremity intervention, and in any venue. It may be used when the extremity is prepped prior to debridement or intervention, during the procedure to help collect debris, fluid, contamination, or after for irrigation, lavage or rinsing. The extremity is placed gently into the basin with distal part of the extremity lying on the extremity resting surface or slope, with the weight of the extremity keeping the basin in place. As the procedure begins, debris, contaminants, fluids (blood, purulence, drainage) are irrigated away with normal or mechanical irrigation or lavage. If no suction is used, the extremity is still protected from the fluid within the basin since it is elevated above on the resting slope.

### BRIEF DESCRIPTION OF THE DRAWINGS

FIG. 1 is a perspective view of a preferred embodiment of the invention;

FIG. 2 is a top view of the embodiment of FIG. 1; and

FIG. 3 is a bottom view of the preferred embodiment;

FIG. 4 is a first side view of the invention;

FIG. 5 is a second side view of the invention;

FIG. 6 is a front view; and

FIG. 7 is a rear view.

### DETAILED DESCRIPTION OF THE PREFERRED EMBODIMENTS

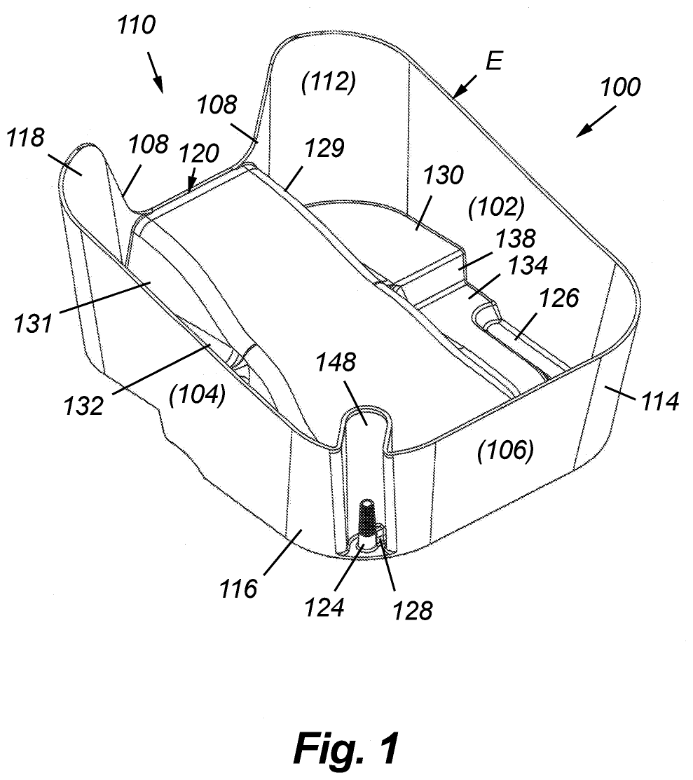

Now making reference to the accompanying drawing, FIG. 1 is a perspective view of a preferred embodiment of the invention, shown generally at 100. The basin is preferably constructed as a unitary, molded, single plastic article using a material such as polypropylene. Wall thickness may be generally uniform, with a thickness on the order of ⅛″ to ¼″ to address desired tradeoffs regarding sturdiness, rigidity, weight and cost.

The article is preferably injection molded or thermoformed. It is important to note that while certain dimensions are presented, these are exemplary only, and may be varied or adjusted in accordance with a desired application. Indeed, the inventive basis may be provided in different sizes (i.e., adult and child), and mechanisms other than those shown may be used to attach basin to appropriate hospital or treatment room furniture. Materials other than polypropylene may likewise be used.

The basin 100 is generally rectangular in shape, with four sidewalls 102, 104, 106, 108 extending upwardly from a closed bottom to an open top. The sidewalls transition to one another through curved corner sections 112, 114, 116, 118. The front of the basin includes an extremity opening or entry channel 110, with the lower edge 120 of the channel 110 preferably smoothly transitioning to a generally downward sloping extremity resting surface 122. The preferred embodiment further includes an integrated suction port 124 in fluid communication with the lowest surface internal to the basin, namely, ring-shaped surface 126. Fluid communication is facilitated by way of a small passageway from the suction port 124 to the lower surface 126.

The basin may include additional internal platforms and sloping surfaces forming a series of troughs at different levels. For example, the forward portion of extremity resting surface 122 transitions to platforms 130, 132 through sloping surfaces 129, 131. Platforms 130, 132 may, in turn, transition to an annular intermediate platform 134 though sloping side surfaces 138, 140. Annular intermediate trough 134 further transitions to bottom ring-shaped trough 126 though sloping surface 144. The lower portion of extremity resting surface 122 transitions to trough 134 through sloping surfaces 123, 125.

The purpose of the various internal platforms, sloping surfaces and troughs is two-fold. First, such structures add to overall rigidity. But secondly, and more importantly, the various levels act to keep the work are as clean as possible during procedures. The upper portion of extremity resting surface 122 smoothly transitions to platforms 130, 132. The lower edge 146 of extremity resting surface 122 smoothly transitions to intermediate trough 134, such that the entire lower portion of extremity resting surface 122 transitions to trough 134. With such a construction, fluids and materials readily flow off extremity resting surface 122 to the various levels, ultimately to trough 126 and suction port 124, keeping each level relatively free of liquids and debris.

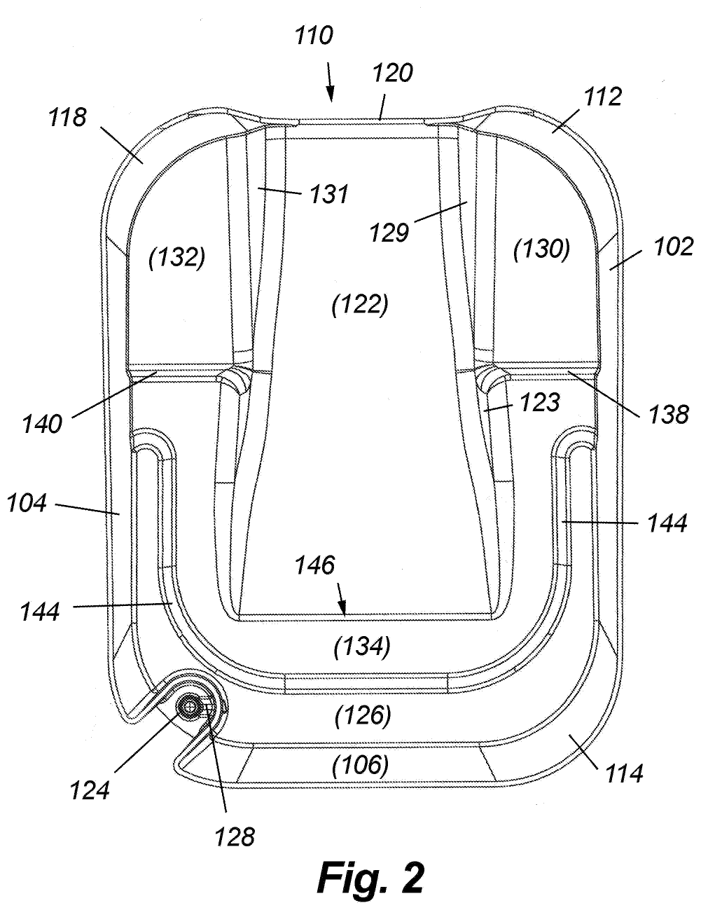

The right and left halves of the basin are generally symmetrical on either side of a central dividing line running from front to back, but for the inclusion of the suction port 124, which is preferably disposed outside the basin proper, surrounded by a semi-circular upwardly extending sidewall 148. FIG. 2 is a top view of the preferred embodiment, perhaps better illustrating all of the various platform levels and sloping surfaces. This figure also better shows passageway 128 from lowest annular surface 126 to suction port 124. Note that the sloping extremity resting surface 122 preferably widens from edge 120 to edge 146 where the bottom portion of surface 122 transitions to intermediate trough 134.

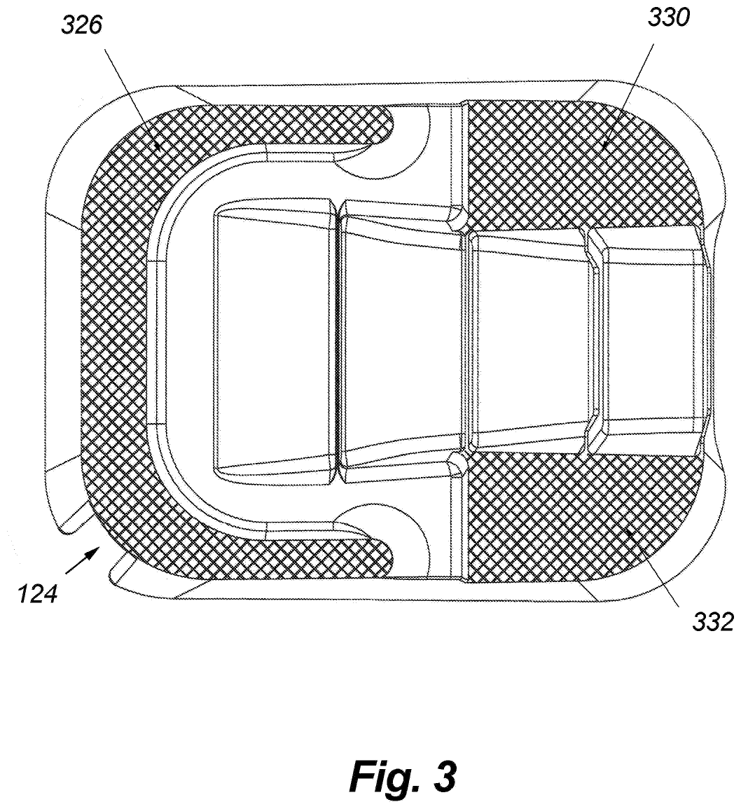

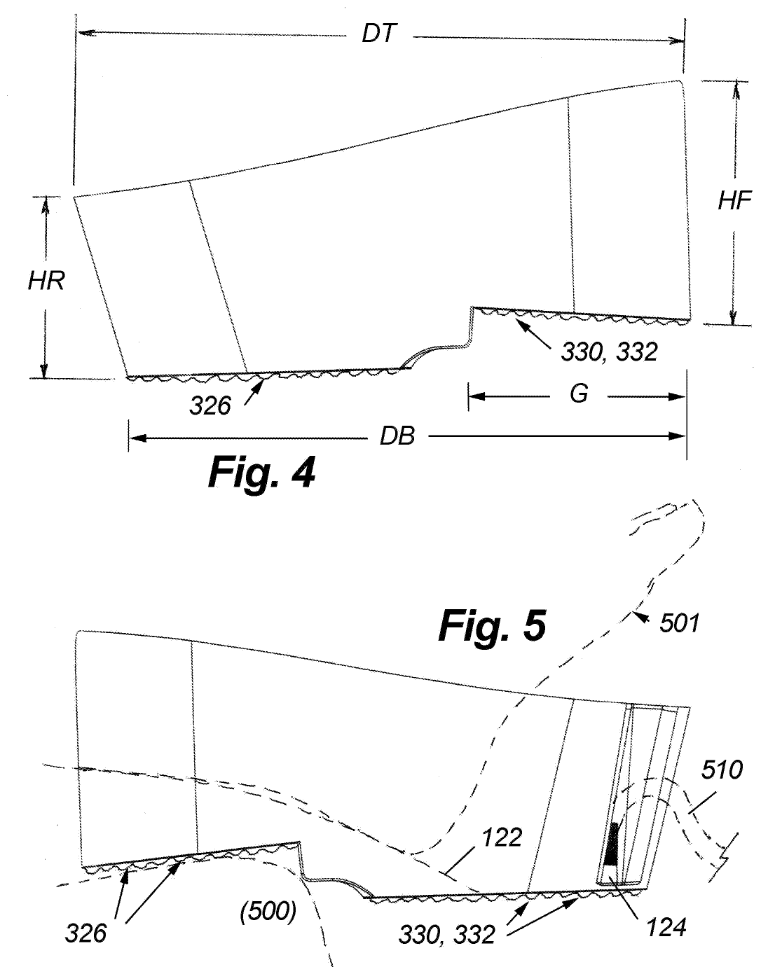

FIG. 3 is a bottom view of the basin showing how particular bottom surfaces preferably include textured surfaces. For example, the bottom outer surfaces 330, 332 associated with side platforms 130, 132 are preferably textured, as is the outer surface 326 associated with the underside of trough 126. These areas include this texturing to assist in maintaining the basin in position when placed onto a bed or other surface, as discussed with reference to FIGS. 4, 5.

FIG. 4 is a first side view of the invention, and FIG. 5 is a view of the opposing side. In terms of dimensions, the depth of the top edge, DT, is preferably in the range of 11 to 15 inches, more preferably in the range of 12 to 14 inches, and most preferably about 13 inches. The depth of the bottom edge, DB, may be in the range of 10 to 14 inches, more preferably in the range of 11 to 13 inches, and most preferably about 12 inches. Note that overall periphery of the bottom is preferably smaller that the overall periphery of the top edge E to ensure that all of the sidewalls slope downwardly and inwardly toward the bottom.

Continuing the reference to FIGS. 4, 5, the height of the rear wall, HR, may be in the range of 2 to 6 inches, more preferably in the range of 3 to 5 inches, and most preferably about 4 inches. The height of the front wall, HF, may be in the range of 3 to 7 inches, more preferably in the range of 4 to 6 inches, and most preferably about 4 inches. It has been discovered through experimentation, that these numbers and ranges most adequately address adult and child extremities while providing for the most efficient irrigation and cleansing procedures. As mentioned, any or all of these dimensions may be varied depending upon the procedure; for example, DT may be made shorter for foot procedures or longer for arm-related operations.

The preferred embodiments further include a step-off groove G that cuts widthwise into the bottom of the basin as shown in FIGS. 4, 5. As shown in FIG. 5, this cut-out anchors the basin to the edge of an OR table, exam chair, exam table, and or any edged examination platform 500. Cross-hatched bottom surfaces 330, 332, 326 enhance stability and grip. Once engaged, the distal portion of the basin declines (by design) and is held in place by the body weight of the extremity 501. As a result, fluids drain into the basin in a “downhill” manner, collect into the series of troughs, to be vacated via integrated suction portal 124. The contaminated fluid then travels via suction through hose 510 into an FDA approved suction canister(s) (not shown), further decreasing contamination. The basin can also be used on any flat surface, does not require suction, and can collect up to 1 liter of irrigation/fluid without spillage.

Prior to this invention, the extremity rests awkwardly on edge or within a simple tray, often causing the distal end of the tray to tip upwards, allowing contaminated fluid to escape. This invention allows the extremity to rest comfortably “inside” the basin on surface 122, within the basin and with a stabile weight distribution and direct orientation via an entry channel that is elevated, thereby decreases the chances of splash or “sloshing” of contaminated fluids on the patient, clinician or treatment area.

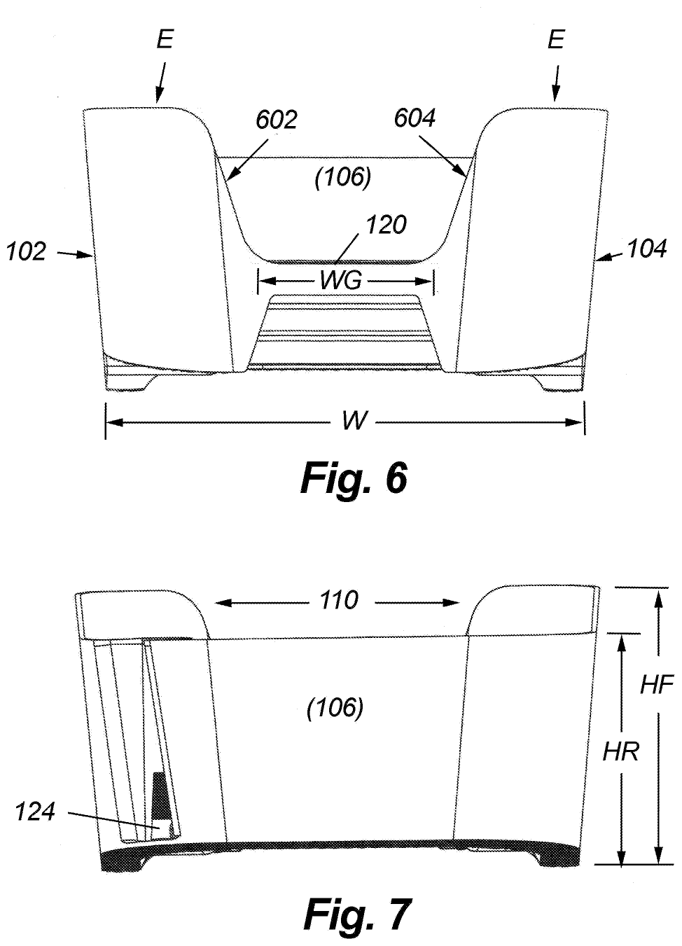

FIG. 6 is a front view of a preferred basin, and FIG. 7 is a rear view. The width W of the lower portion of the basin may be in the range 8 to 12 inches, more preferably in the range of 9 to 11 inches, and most preferably 10 inches, more or less. The width of the upper portion is at least slightly larger to achieve sloping sidewalls 102, 104. The width WG of the bottom edge 120 of the extremity opening or entry channel 110 may be in the range of 2 to 5 inches, more preferably in the range of 2.5 to 4.5 inches, and most preferably 3.5 inches, more or less. The sides 602, 604 of the gap preferably slope inwardly and downwardly from the upper edge E, with rounded corners throughout to enhance comfort.

1. An article for irrigating extremities, comprising:
a basin having a bottom panel with an upper surface, an underside, and a periphery;
a sidewall that extends upwardly from the periphery of the bottom panel to an upper edge that defines an open top into a basin interior;
wherein the basin has a front portion, a rear portion and opposing side portions, and wherein the sidewall associated with the front portion of the basin includes a gap adapted to receive an extremity;
a downwardly sloping ramp forming an extremity resting surface that extends from the gap and into the basin;
wherein the downwardly sloping ramp transitions into sloping side surfaces to a lower tough at the bottom of the basin; and
an integrated suction port in fluid communication with the lower trough.

2. The article of claim 1, further including a widthwise cut-out in the underside of the front portion of the basin to engage with the upper edge of a table, bed or other furniture to maintain the basin in position during use thereof.

3. The article of claim 1, wherein at least the underside of the front portion of the basin includes a textured surface to enhance frictional gripping.

4. The article of claim 1, further including at least one intermediate trough at a level between the downwardly sloping ramp and the lower tough at the bottom of the basin.

5. The article of claim 1, wherein the basin is constructed as a unitary molded plastic article.

6. The article of claim 1, wherein the basin is disposable after use.
